(19) United States
(12) Patent Application Publication
(10) Pub. No.: US 2014/0170058 A1
(43) Pub. Date: Jun. 19, 2014

(54) PROCESS FOR THE REMOVAL OF PALLADIUM FROM 4-AMINO-3-HALO-5-FLUORO-6-(ARYL) PYRIDINE-2-CARBOXYLATES AND 4-AMINO-3-HALO-6-(ARYL)PYRIDINE-2-CARBOXYLATES

(71) Applicants: Jossian Oppenheimer, Midland, MI (US); Mark V. M. Emonds, Midland, MI (US)

(72) Inventors: Jossian Oppenheimer, Midland, MI (US); Mark V. M. Emonds, Midland, MI (US)

(73) Assignee: DOW AGROSCIENCES LLC, INDIANAPOLIS, IN (US)

(21) Appl. No.: 13/828,032

(22) Filed: Mar. 14, 2013

Related U.S. Application Data
(60) Provisional application No. 61/736,828, filed on Dec. 13, 2012.

(51) Int. Cl. C07D 213/79; C22B 3/00

(52) U.S. Cl. CPC C07D 213/79; C22B 11/04
USPC 423/519.2; 546/309; 546/310

(57) ABSTRACT

Residual palladium is removed and recovered from 4-amino-3-halo-5-fluoro-6-(aryl)pyridine-2-carboxylates and 4-amino-3-halo-6-(aryl)pyridine-2-carboxylates by treatment with an aqueous solution containing from about 20 to about 50% of an alkali metal bisulfite at a temperature from about 60 to about 90° C. and a pH from about 3.8 to about 7.0.

This application claims the benefit of U.S. Provisional Patent Application No. 61/736,828, filed Dec. 13, 2012, the entirety of which is incorporated herein by reference.

### BACKGROUND

Provided herein are improved processes for the removal of palladium from protected or unprotected 4-amino-3-halo-5-fluoro-6-(aryl)pyridine-2-carboxylates and 4-amino-3-halo-6-(aryl)pyridine-2-carboxylates.

U.S. Pat. Nos. 6,784,137 B2 and 7,314,849 B2 describe inter alia certain 4-amino-3-halo-5-fluoro-6-(aryl)pyridine-2-carboxylate and 4-amino-3-halo-6-(aryl)pyridine-2-carboxylate compounds and their use as herbicides. More often than not, these herbicides are prepared from coupling reactions involving pyridine-2-carboxylate having either a facile leaving group or a metal derivative in the 6-position of the pyridine ring. Such coupling reactions usually employ a transition metal catalyst, in particular a palladium catalyst such as palladium diacetate or bis(triphenylphosphine)palladium(II) dichloride. For reasons of both product stewardship and economics, it is important to remove and recover the palladium used in the manufacturing process from the herbicide product.

K. M. Bullock, M. B. Mitchell and J. F. Toczko, Org. Process Res. Dev., 2008, 12 (5), 896-899 describe the reduction in palladium levels from 12,000 ppm to about 100 ppm in a pharmaceutical product made by a Suzuki-Miyaura coupling by treating the reaction mixture using a toluene extraction—sodium bisulfite wash procedure in which the reaction mixture, with added toluene, is treated with 20% NaHSO₃ at 60° C. for 1 hour. When this procedure is applied to protected or unprotected 4-amino-3-halo-5-fluoro-6-(aryl)pyridine-2-carboxylate and 4-amino-3-halo-6-(aryl)pyridine-2-carboxylate herbicides, however, palladium levels are found to be reduced from about 1,500 ppm to only about 600 ppm. It would be desirable to have a process that reduces residual palladium levels in protected or unprotected 4-amino-3-halo-5-fluoro-6-(aryl)pyridine-2-carboxylate and 4-amino-3-halo-6-(aryl)pyridine-2-carboxylate herbicides to less than 100 ppm. It would also be desirable to be able to efficiently recover the palladium from the process.

### SUMMARY

Provided herein are processes for the removal of residual palladium from the manufacture of protected or unprotected 4-amino-3-halo-5-fluoro-6-(aryl)pyridine-2-carboxylate and 4-amino-3-halo-6-(aryl)pyridine-2-carboxylate herbicides by a palladium-catalyzed coupling reaction. More particularly, provided herein are processes for the removal of residual palladium from the manufacture of a compound of Formula I

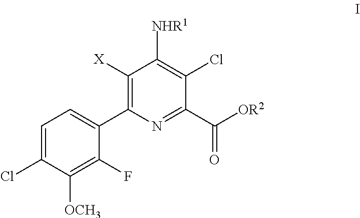

in which
X represents H or F,
R¹ represents H or —C(O)CH₃, and
R² represents C₁-C₁₂ alkyl or an unsubstituted or substituted C₇-C₁₁ arylalkyl
   by a palladium-catalyzed coupling reaction which comprises (a) contacting the compound of Formula I in a water-immiscible solvent with an aqueous solution containing from about 20 to about 50% of an alkali metal bisulfite at a temperature from about 60 to about 90° C. and a pH from about 3.8 to about 7.0; (b) separating the aqueous phase from the water-immiscible solvent phase; and (c) recovering the compound of Formula I from the water-immiscible solvent phase. Another aspect concerns recovering the residual palladium.

### DETAILED DESCRIPTION

The term “alkyl” and derivative terms such as “alkoxy,” as used herein refer to straight chain or branched chain moieties. Typical C₁-C₁₂ alkyl groups are methyl, ethyl, propyl, 1-methylethyl, butyl, 1,1-dimethylethyl, 1-methylpropyl, 1-methylhexyl, 2-ethylhexylm, 1-methylheptyl and dodecyl. Methyl and ethyl are often preferred.

The term “arylalkyl,” as used herein, refers to a phenyl substituted alkyl group having a total of 7 to 11 carbon atoms, such as benzyl (—CH₂C₆H₅), 2-methylnaphthyl (—CH₂C₁₀H₇) and 1- or 2-phenethyl (—CH₂CH₂C₆H₅ or —CH(CH₃)C₆H₅). The phenyl group may itself be unsubstituted or substituted with one or more substituents independently selected from halogen, nitro, cyano, C₁-C₆ alkyl, C₁-C₆ alkoxy, halogenated C₁-C₆ alkyl, halogenated C₁-C₆ alkoxy, C₁-C₆ alkylthio, C(O)OC₁-C₆ alkyl, or where two adjacent substituents are taken together as —O(CH₂)ₙO— wherein n=1 or 2, provided that the substituents are sterically compatible and the rules of chemical bonding and strain energy are satisfied.

Alkali metals refer to members of groups 1 of the periodic table. Preferred alkali metal (group 1) salts are sodium and potassium salts.

Protected 4-amino-3-halo-5-fluoro-6-(aryl)pyridine-2-carboxylate and 4-amino-3-halo-6-(aryl)pyridine-2-carboxylate compounds refer to compounds in which the 4-amino group is protected by an acetyl group.

Protected or unprotected 4-amino-3-halo-5-fluoro-6-(aryl)pyridine-2-carboxylate and 4-amino-3-halo-6-(aryl)pyridine-2-carboxylate compounds are prepared from coupling reactions involving pyridine-2-carboxylate having either a facile leaving group or a metal derivative in the 6-position of the pyridine ring. Such coupling reactions usually employ a transition metal catalyst, in particular a palladium catalyst such as palladium diacetate or bis(triphenylphosphine)palladium(II) dichloride (see U.S. Pat. Nos. 6,784,137 B2 and 7,314,849 B2). After conventional work-up procedures, the isolated products typically contain between 900 and 1,500 ppm of residual palladium species.

For the palladium-removal process, the protected or unprotected 4-amino-3-halo-5-fluoro-6-(aryl)pyridine-2-carboxylate and 4-amino-3-halo-6-(aryl)pyridine-2-carboxylate compounds are dissolved in a water immiscible solvent. Preferred water immiscible solvents are those which can also serve as solvents for the coupling reaction itself and include, for example, aromatic hydrocarbons such as toluene or xylene and solvent mixtures such as 4-methyl-2-pentanone, 1,2-dimethoxyethane and acetonitrile. The water immiscible solvent phase typically contains from about 10 to about 30% of the protected or unprotected 4-amino-3-halo-5-fluoro-6-(aryl)pyridine-2-carboxylate or 4-amino-3-halo-6-(aryl)pyridine-2-carboxylate compound.

The protected or unprotected 4-amino-3-halo-5-fluoro-6-(aryl)pyridine-2-carboxylate or 4-amino-3-halo-6-(aryl)pyridine-2-carboxylate compound in the water-immiscible solvent is treated with from about 1 to about 6 molar equivalents of an aqueous solution containing from about 20 to about 50% of an alkali metal bisulfite at a temperature from about 60 to about 90° C. and a pH from about 3.8 to about 7.0. Preferably, from about 1 to about 2 molar equivalents of the aqueous solution containing about 20-30% of an alkali metal bisulfite at a temperature of about 80° C. and a pH of about 5.4 is employed. The pH is routinely controlled by the addition of sodium hydroxide, most conveniently by the use of 20-50% aqueous NaOH.

After separating the aqueous phase from the water-immiscible solvent phase, the protected or unprotected 4-amino-3-halo-5-fluoro-6-(aryl)pyridine-2-carboxylate or 4-amino-3-halo-6-(aryl)pyridine-2-carboxylate compound is recovered from the water-immiscible solvent phase by a crystallization process. The recovered protected or unprotected 4-amino-3-halo-5-fluoro-6-(aryl)pyridine-2-carboxylate or 4-amino-3-halo-6-(aryl)pyridine-2-carboxylate contains less the 100 ppm of palladium, and in some embodiments, less than 90 ppm.

Sodium bisulfite forms an insoluble palladium tetrasulfite dihydrate complex with the residual palladium in the protected or unprotected 4-amino-3-halo-5-fluoro-6-(aryl)pyridine-2-carboxylate or 4-amino-3-halo-6-(aryl)pyridine-2-carboxylate compound. This complex can be recovered by filtration and the palladium can be reclaimed by means well known to those of ordinary skill in the art.

The described embodiments and following examples are for illustrative purposes and are not intended to limit the scope of the claims. Other modifications, uses, or combinations with respect to the compositions described herein will be apparent to a person of ordinary skill in the art without departing from the spirit and scope of the claimed subject matter.

### EXAMPLES

### Example 1

### Synthesis of methyl 4-(acetylamino)-3-chloro-6-(4-chloro-2-fluoro-3-methoxyphenyl)pyridine-2-carboxylate

To a 1-L jacketed reactor equipped with a condenser, mechanical stirrer, thermocouple temperature probe, and nitrogen inlet was added 40.0 g of methyl 4-(acetylamino)-3,6-dichloropyridine-2-carboxylate, 0.4 g of triphenylphosphine, and 0.17 g of palladium(II) acetate under a nitrogen atmosphere. A 25 ml rinse with acetonitrile (previously degassed by sparging with nitrogen for 45 minutes) was employed to ensure all solids were transferred to the reactor. A total of 157.1 g of a 22% solution of 4-chloro-2-fluoro-3-methoxyphenylboronic acid (previously degassed by sparging with nitrogen for 45 minutes) in 1,2-dimethoxyethane (DME) and methyl isobutyl ketone (MIBK) was added under nitrogen via a pump. The ratio of MIBK to DME in the solution varies from 2:1 to 0.7:1 (MIBK:DME) depending on the equivalents of the solvents used to prepare the 4-chloro-2-fluoro-3-methoxyphenylboronic acid solution, or more commonly from 1.6:1 to 1.2:1. Stirring at 300 RPM was initiated. An additional 100 ml of acetonitrile (previously degassed by sparging with nitrogen for 45 minutes) was added and complete dissolution was achieved. A total of 275.3 g of a 22.9% aqueous solution of potassium carbonate (previously degassed by sparging with nitrogen for 45 minutes) was added via a pump. The solution was heated to about 50° C. for 2.5 hours. Towards the end of the reaction, the product tended to precipitate and a small product rind formed on the reactor wall. The organic phase of the mixture was sampled and analyzed by GC to determine reaction completion.

The mixture was heated to about 65° C. to ensure complete product dissolution. The mixture was filtered (polish filtration) at 65° C. to remove reduced palladium and inorganic salts. The organic and aqueous phases were separated at 65° C.

### Example 2

### Removal of residual palladium from methyl 4-(acetylamino)-3-chloro-6-(4-chloro-2-fluoro-3-methoxyphenyl)pyridine-2-carboxylate

Sixty-eight grams of the organic stream of the Suzuki coupling reaction (Example 1; 13 g of methyl 4-(acetylamino)-3-chloro-6-(4-chloro-2-fluoro-3-methoxyphenyl)pyridine-2-carboxylate, 1100 ppm palladium level in the solid) was warmed up to 60° C. using a water bath and transferred to a 250 mL reactor equipped with a water condenser, thermocouple temperature probe and a mechanical stirrer. A pH 5.4 (adjusted with sodium hydroxide) 30 wt % sodium bisulfite solution (93 mL) was added to the flask. The mixture was heated to 80° C., and held at that temperature for 6 hr. After 6 hr, the aqueous phase was drained and filtered through a Whatman #1 filter paper using a preheated filtration flask and a Buchner funnel. The hot organic phase was transferred to a heated bottle and 6 mL of organic solution was filtered using 0.45 um syringe filter. The filtered organic phase was concentrated to dryness and further dried in a vacuum oven at 55° C. The dried solid was submitted for palladium analysis using neutron activation analysis. The palladium content in the product was 82 ppm.

What is claimed is:

1. A process for the removal of residual palladium from the manufacture of a compound of Formula I 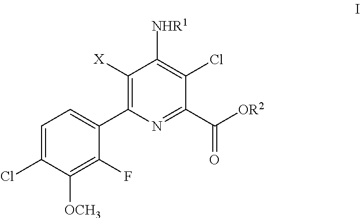
in which
X represents H or F,
R¹ represents H or —C(O)CH₃, and
R² represents C₁-C₁₂ alkyl or an unsubstituted or substituted C₇-C₁₁ arylalkyl
by a palladium-catalyzed coupling reaction which comprises
(a) contacting the compound of Formula I in a water-immiscible solvent with an aqueous solution containing from about 20 to about 50% of an alkali metal bisulfite at a temperature from about 60 to about 90° C. and a pH from about 3.8 to about 7.0;
(b) separating the aqueous phase from the water-immiscible solvent phase; and
(c) recovering the compound of Formula I from the water-immiscible solvent phase.

2. A process for the recovery of residual palladium from the manufacture of a compound of Formula I 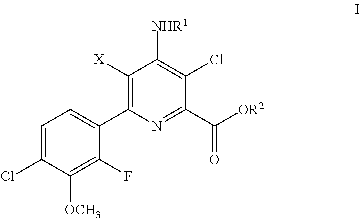
in which
X represents H or F,
R¹ represents H or —C(O)CH₃, and
R² represents C₁-C₁₂ alkyl or an unsubstituted or substituted C₇-C₁₁ arylalkyl
by a palladium-catalyzed coupling reaction which comprises
(a) contacting the compound of Formula I in a water-immiscible solvent with an aqueous solution containing from about 20 to about 50% of an alkali metal bisulfite at a temperature from about 60 to about 90° C. and a pH from about 3.8 to about 7.0;
(b) separating the aqueous phase from the water-immiscible solvent phase; and
(c) recovering the residual palladium from the aqueous alkali metal bisulfite phase.
